(19) United States
(12) Patent Application Publication
(10) Pub. No.: US 2026/0249062 A1
(43) Pub. Date: Aug. 27, 2026

(54) MEDICATION DELIVERY DEVICE FOR RECTAL DELIVERY OF MEDICATION AND METHOD THEREFOR

(71) Applicant: Esther Kumenda, Burnsville, MN (US)

(72) Inventor: Esther Kumenda, Burnsville, MN (US)

(21) Appl. No.: 19/059,489

(22) Filed: Feb. 21, 2025

(51) Int. Cl. A61M 31/00

(52) U.S. Cl. CPC A61M 31/007; A61M 2202/0007; A61M 2202/04; A61M 2202/06; A61M 2205/0216; A61M 2210/1064

(57) ABSTRACT

A medication delivery device for rectal delivery of medication includes a barrel, a tip, and a plunger. The barrel consists of rigid material. The tip has a first tip portion that is cylindrical and a second tip portion is in the form of a truncated cone. The second tip portion includes a discharge orifice that has a diameter smaller than the diameter of the first tip portion. The tip consists of a flexible, stretchable material. The plunger is insertable into the barrel through the opening in the second barrel end. The plunger includes a plunger shaft, a plunger head, and a plunger handle. The plunger head is disk shaped. The plunger handle is T-shaped and positioned perpendicular to the plunger shaft.

### STATEMENT REGARDING FEDERALLY SPONSORED RESEARCH OR DEVELOPMENT

Not Applicable

### CROSS-REFERENCE TO RELATED APPLICATIONS

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to delivery devices for medication and more particularly pertains to a new medication delivery device for delivering different types of medication rectally. Often it can be preferable or necessary to deliver medications, such as creams, gels, tablets, pills, and liquids, rectally rather than by another bodily orifice or through the skin. To facilitate such delivery, syringe-like devices can be used that are inserted into the rectum.

### (2) Description of Related Art including information disclosed under 37 CFR 1.97 and 1.98

The prior art relates to delivery devices for delivering medication rectally. The prior art, as best understood, does not disclose medication delivery device for rectal delivery of medication includes a barrel, a tip, and a plunger, wherein the barrel is made of rigid material and the tip is made flexible, stretchable material, which tip has a discharge orifice that has a diameter smaller than the diameter of the barrel, and wherein the plunger includes a plunger handle that is T-shaped and positioned perpendicular to a plunger shaft.

### BRIEF SUMMARY OF THE INVENTION

An embodiment of the disclosure meets the needs presented above in a medication delivery device for rectal delivery of medication generally comprising a barrel, a tip, and a plunger. The barrel consists of rigid material. The barrel has a first barrel end and a second barrel end. Each of the first barrel end and the second barrel end includes an opening. The tip has a first tip portion coupled to the first barrel end and a second tip portion integrally formed with the first tip portion. The first tip portion is cylindrical and has a diameter the same as a diameter of the barrel. The second tip portion is in the form of a truncated cone. The second tip portion includes a discharge orifice that has a diameter smaller than the diameter of the first tip portion. The tip consists of a flexible, stretchable material such that the second tip portion is expandable for passage of items larger than the diameter of the discharge orifice. The plunger is insertable into the barrel through the opening in the second barrel end. The plunger includes a plunger shaft, a plunger head, and a plunger handle. The plunger shaft has a first shaft end and a second shaft end. The plunger head is attached to the first shaft end. The plunger head is disk shaped. The plunger head has a diameter similar to an inner diameter of the barrel to push medications through the barrel and the tip and out of the discharge orifice. The plunger handle is attached to the second shaft end. The plunger handle is T-shaped and positioned perpendicular to the plunger shaft.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a medication delivery device according to an embodiment of the disclosure.

FIG. 2 is a side view of an embodiment of the disclosure.

FIG. 3 is a rear view of an embodiment of the disclosure.

FIG. 4 is a front view of an embodiment of the disclosure.

FIG. 5 is a side view of an embodiment of the disclosure in use.

FIG. 6 is a cross-sectional view of the embodiment of the disclosure in FIG. 5.

FIG. 7 is a cross-sectional view of the embodiment of the disclosure in FIG. 5.

### DETAILED DESCRIPTION OF THE INVENTION

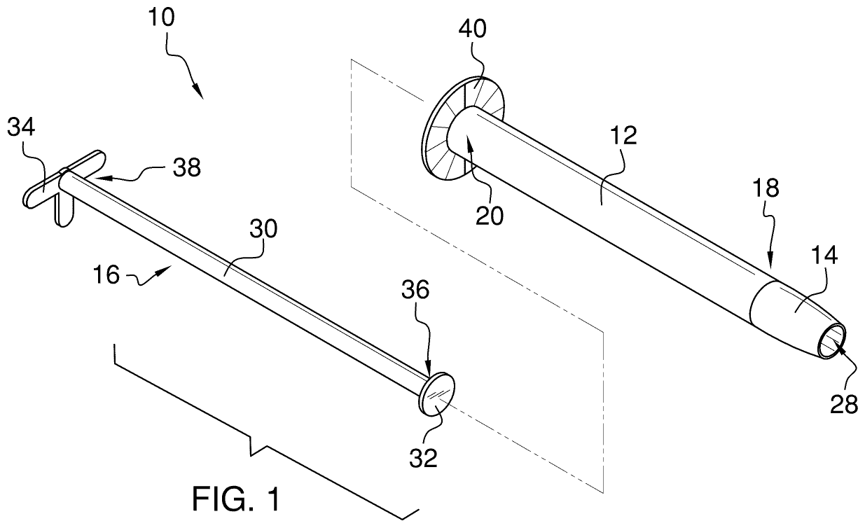

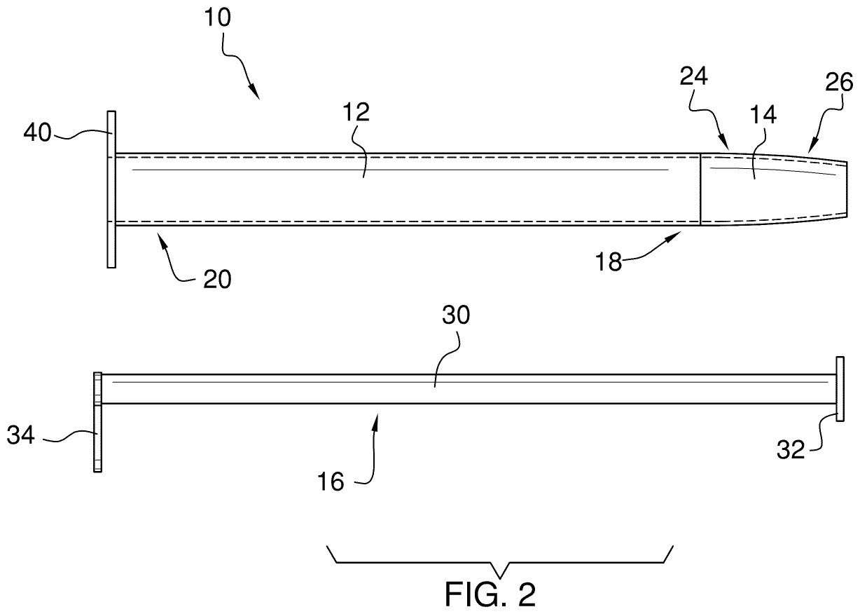

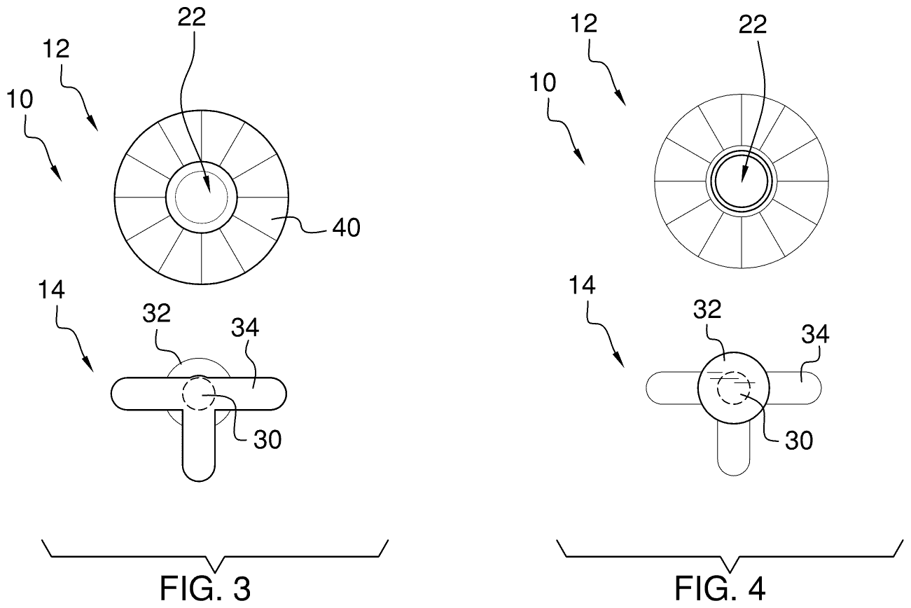

With reference now to the drawings, and in particular to FIGS. 1 through 7 thereof, a new medication delivery device embodying the principles and concepts of an embodiment of the disclosure and generally designated by the reference numeral 10 will be described.

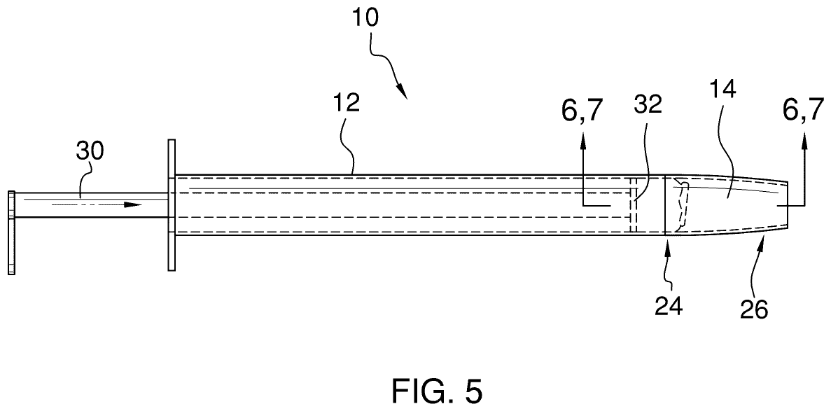

As best illustrated in FIGS. 1 through 7, the medication delivery device 10 generally comprises a barrel 12, a tip 14, and a plunger 16. The barrel 12 consists of rigid material. The barrel 12 has a first barrel end 18 and a second barrel end 20. Each of the first barrel end 18 and the second barrel end 20 includes an opening 22. The tip 14 has a first tip portion 24 coupled to the first barrel end 18 and a second tip portion 26 integrally formed with the first tip portion 24. The first tip portion 24 is cylindrical and has a diameter the same as a diameter of the barrel 12. The second tip portion 26 is in the form of a truncated cone. The second tip portion 26 includes a discharge orifice 28 that has a diameter smaller than the diameter of the first tip portion 24. The tip 14 consists of a flexible, stretchable material such that the second tip portion 26 is expandable for passage of items larger than the diameter of the discharge orifice 28. As shown in FIG. 5, the plunger 16 is insertable into the barrel 12 through the opening 22 in the second barrel end 20. The plunger 16 includes a plunger shaft 30, a plunger head 32, and a plunger handle 34. The plunger shaft 30 has a first shaft end 36 and a second shaft end 38. The plunger head 32 is attached to the first shaft end 36. The plunger head 32 is disk shaped. The plunger head 32 has a diameter similar to an inner diameter of the barrel 12 to push medications through the barrel 12 and the tip 14 and out of the discharge orifice 28. The plunger handle 34 is attached to the second shaft end 38. The plunger handle 34 is T-shaped and positioned perpendicular to the plunger shaft 30.

In the exemplary embodiment, an annular flange 40 is attached to the second end of the barrel 12. The annular flange 40 is disk-shaped. The annular flange 40 can be integrally formed as a one-piece structure with the barrel 12.

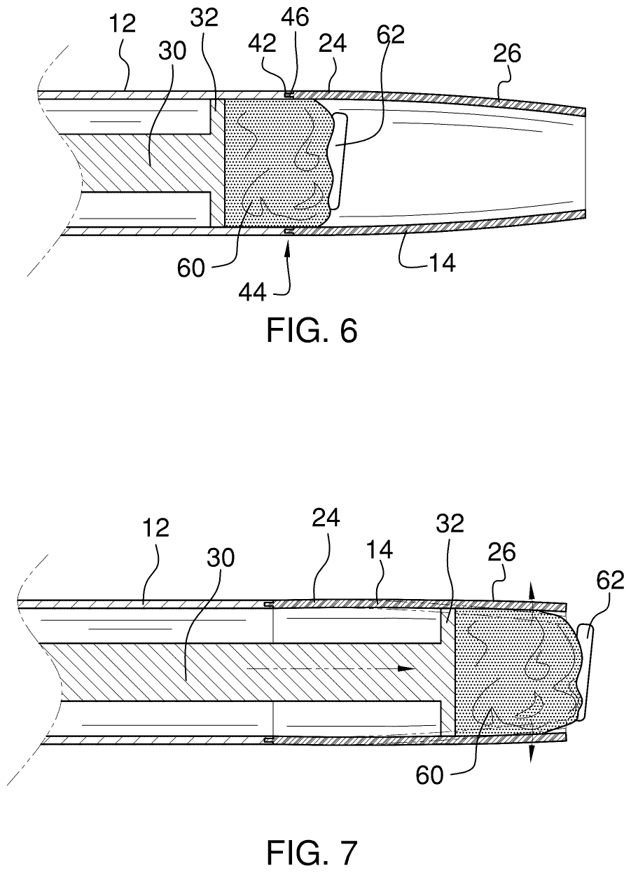

As best seen in FIGS. 6 and 7, the barrel 12 includes a groove 42 in a rim 44 about the opening 22 in the second barrel end 20. The tip 14 includes a ring 46 projecting from the first tip portion 24. The ring 46 is inserted into the groove 42 to couple the tip 14 to the barrel 12. In one possible embodiment, the tip 14 is removable and replaceable with a new one. In another possible embodiment, the tip 14 is permanently attached to the barrel 12, such as by adhesive.

In the exemplary embodiment, the barrel 12 consists of medical-grade plastic, though in other possible embodiments other materials, such as metal, could be used. In the exemplary embodiment, the tip 14 consists of silicone. Other stretchable materials could be used, such as other types of plastics or elastomers.

The plunger shaft 30 and the plunger head 32 together are at least as long as the barrel 12 and the tip 14 combined to ensure complete discharge of medications out through the discharge orifice 28. In one possible embodiment, the plunger shaft 30 and the plunger head 32 together are longer than the barrel 12 and the tip 14 combined such that the plunger head 32 can protrude out of the tip 14 to ensure complete discharge of medications out through the discharge orifice 28. In the exemplary embodiment, the barrel 12 is at least 10.0 centimeters long, though other sizes are within the scope of the disclosure.

As shown in FIGS. 6 and 7, the medication delivery device 10 is used for rectally delivering medication. Medication is inserted into at least one of the barrel 12 and the tip 14. The tip 14 is then inserted into a rectum (not shown), which insertion is facilitated by the tapered design of the tip 14. The user then grasps the plunger handle 34 and pushes the plunger 16 to cause the plunger head 32 to engage and push the medication out of the discharge opening 22 and into the rectum. The medication itself can be of different types and sizes, such as semi-solid medications like creams and gels, solid medications like tablets and capsules, and liquid medications like oils and solutions. In the exemplary embodiment shown in FIGS. 6 and 7, a combination of a cream 60 and a tablet 62 is in the barrel 12 and in the tip 14. The tablet 62 has a diameter that is slightly larger than the diameter of the discharge orifice 28, as does the volume of cream 60. As shown in FIG. 7, the discharge opening 22 stretches when pushing the medication through the discharge opening 22 to allow the cream 60 and the tablet 62 to exit the discharge opening 22. Because of the size of the medication delivery device 10 and the stretchable design of the tip 14, larger medications and more than one medication can be delivered at the same time.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medication delivery device for rectal delivery of medication comprising:
a barrel consisting of rigid material, said barrel having a first barrel end and a second barrel end, each of said first barrel end and said second barrel end including an opening;
a tip having a first tip portion coupled to said first barrel end and a second tip portion integrally formed with said first tip portion, said first tip portion being cylindrical and having a diameter the same as a diameter of said barrel, said second tip portion being in the form of a truncated cone, said second tip portion including a discharge orifice having a diameter smaller than the diameter of said first tip portion, said tip consisting of flexible, stretchable material such that said second tip portion is expandable for passage of items larger than the diameter of said discharge orifice; and
a plunger being insertable into said barrel through said opening in said second barrel end, said plunger including:
a plunger shaft having a first shaft end and a second shaft end;
a plunger head being attached to said first shaft end, said plunger head being disk shaped, said plunger head having a diameter similar to an inner diameter of said barrel to push medications through said barrel and said tip and out of said discharge orifice; and
a plunger handle being attached to said second shaft end, said plunger handle being T-shaped and positioned perpendicular to said plunger shaft.

2. The rectal medication delivery device of claim 1, further comprising an annular flange being attached to said second end of said barrel.

3. The rectal medication delivery device of claim 2, wherein said annular flange is disk-shaped.

4. The rectal medication delivery device of claim 1, wherein:
said barrel includes a groove in a rim about said opening in said second barrel end;
said tip includes a ring projecting from said first tip portion; and
said ring is inserted into said groove to couple said tip to said barrel.

5. The rectal medication delivery device of claim 1, wherein said barrel consists of medical-grade plastic.

6. The rectal medication delivery device of claim 1, wherein said tip consists of silicone.

7. The rectal medication delivery device of claim 1, wherein said plunger shaft and said plunger head together are at least as long as said barrel and said tip combined to ensure complete discharge of medications out through said discharge orifice.

8. The rectal medication delivery device of claim 7, wherein said barrel is at least 10.0 centimeters long.

9. The rectal medication delivery device of claim 3, wherein:
said barrel includes a groove in a rim about said opening in said second barrel end;
said tip includes a ring projecting from said first tip portion; and
said ring is inserted into said groove to couple said tip to said barrel.

10. The rectal medication delivery device of claim 9, wherein said barrel consists of medical-grade plastic.

11. The rectal medication delivery device of claim 10, wherein said tip consists of silicone.

12. The rectal medication delivery device of claim 11, wherein said plunger shaft and said plunger head together are at least as long as said barrel and said tip combined to ensure complete discharge of medications out through said discharge orifice.

13. The rectal medication delivery device of claim 12, wherein said barrel is at least 10.0 centimeters long.

14. A method of rectally delivering medication, said method comprising the steps of:
providing a medication delivery comprising:
a barrel consisting of rigid material, said barrel having a first barrel end and a second barrel end, each of said first barrel end and said second barrel end including an opening;
a tip having a first tip portion coupled to said first barrel end and a second tip portion integrally formed with said first tip portion, said first tip portion being cylindrical and having a diameter the same as a diameter of said barrel, said second tip portion being in the form of a truncated cone, said second tip portion including a discharge orifice having a diameter smaller than the diameter of said first tip portion, said tip consisting of flexible, stretchable material such that said second tip portion is expandable for passage of items larger than the diameter of said discharge orifice; and
a plunger being insertable into said barrel through said opening in said second barrel end, said plunger including:
a plunger shaft having a first shaft end and a second shaft end;
a plunger head being attached to said first shaft end, said plunger head being disk shaped, said plunger head having a diameter similar to an inner diameter of said barrel to push medications through said barrel and said tip and out of said discharge orifice; and
a plunger handle being attached to said second shaft end, said plunger handle being T-shaped and positioned perpendicular to said plunger shaft;
inserting a medication into at least one of said barrel and said tip;
inserting said tip into a rectum; and
grasping said plunger handle and pushing said plunger to cause said plunger head to engage and push the medication out of said discharge opening and into the rectum.

15. The method of claim 14, wherein the medication includes a solid material having a diameter larger than the diameter of said discharge opening, and said method further comprises stretching said discharge opening and pushing the medication through said discharge opening.

16. The method of claim 15, wherein:
said step of inserting a medication includes inserting at least two different ones of a solid medication, a semi-solid medication, and a liquid medication; and
said step of pushing said plunger includes pushing all of the medications into the rectum.
